(19) United States
(12) Patent Application Publication
(10) Pub. No.: US 2015/0056273 A1
(43) Pub. Date: Feb. 26, 2015

(54) MICROBUBBLE ULTRASOUND CONTRAST AGENT FOR EXTERNAL USE

(71) Applicants: National Taiwan University of Science and Technology, Taipei (TW); National Defense Medical Center, Taipei (TW)

(72) Inventors: Ai-Ho LIAO, TAIPEI CITY (TW); Chih-Hung WANG, TAIPEI CITY (TW); Yi-Lei HSIEH, TAIPEI CITY (TW); Hsin-Chiao HO, TAIPEI CITY (TW)

(21) Appl. No.: 14/526,496

(22) Filed: Oct. 28, 2014

Related U.S. Application Data
(63) Continuation-in-part of application No. 13/961,903, filed on Aug. 8, 2013.

(30) Foreign Application Priority Data
Jun. 25, 2013 (TW) 102122588

(51) Int. Cl. A61K 9/50; A61K 47/26; A61K 47/42

(52) U.S. Cl. CPC A61K 9/50; A61K 47/42; A61K 47/26
USPC 424/450; 424/489

(57) ABSTRACT

A microbubble ultrasound contrast agent for external use is provided. The microbubble ultrasound contrast agent applied externally can safely and efficiently enhance the permeation and absorption of the drug or small molecules in the local region of the body surface. A method of preparing the microbubble ultrasound contrast agent and a method of enhancing percutaneous absorption of a chemical or small molecules through a topical region of a biological body surface are provided.

### CROSS-REFERENCE TO RELATED APPLICATION

The present application is a continuation-in-part application of U.S. application Ser. No. 13/961,903, filed Aug. 8, 2013 and claims priority to Taiwan Application Serial Number 102122588, filed Jun. 25, 2013, which is herein incorporated by reference.

### BACKGROUND

1. Technical Field

The present invention relates to a biomedical agent. Particularly, the present invention relates to an ultrasound microbubble contrast agent for external uses.

2. Related Art

For decades, ultrasound has been one of the most important tools in the medical or therapeutic field as it is an accurate, inexpensive and easily operated tool with no ionizing radiation. For the ultrasonic technology, the microbubble ultrasound contrast agent is applied intravascularly and the tiny bubbles of the microbubble ultrasound contrast agent in the blood vessel are excited by ultrasonic energy to generate harmonic resonance, which enhances the received ultrasound images. The application of the microbubble ultrasound contrast agents may help increase the contrast resolution and sensitivity of high-frequency ultrasound imaging. However, as the conventional microbubble ultrasound contrast agent is injected into the blood vessel or into the living body, an overall risk of applying the conventional microbubble ultrasound contrast agent is somehow higher, which is detrimental for medical or research applications.

### SUMMARY

The present invention provides an external type microbubble ultrasound contrast agent of topical uses. The microbubble ultrasound contrast agent may be applied to a topical region of the body surface of the living body by coating, instead of using injection. The external type microbubble ultrasound contrast agent may employ a medium, either aqueous or a gel form, and contain microbubbles of a specific particle size and at a specific concentration. The material of the microbubbles may be albumin, liposomes, polymers, copolymers or mixtures of the aforementioned material or a combination of those above. The material of the microbubbles may also include pentose and/or hexose. A method for preparing an external type microbubble ultrasound contrast agent of topical uses is also provided. The microbubble ultrasound contrast agent may have microbubbles with different particle sizes by adjusting the percentage of the medium and the material of the microbubbles in the mixed solution and followed by the same ultrasonic oscillating steps which oscillating the mixed solution for about 100 to about 140 seconds. The external type microbubble ultrasound contrast agent may be applied in conjunction with the application of mechanical oscillation waves. Through a series of swelling and shrinking processes induced by the oscillation energy of the mechanical oscillation waves, the microbubbles burst or destructed and the generated energy and shock waves lead to minor damages of cells or tissues, which further strengthen the absorption of applied chemicals or small molecules, also the microbubbles with different particle sizes may lead to different penetration depth for the applied chemicals or small molecules. The commonly used energy source of the mechanical oscillation waves may be a source of an optical energy or acoustic energy, such as an ultrasound source or a laser source. The external type microbubble ultrasound contrast agent of the present invention, suitable for applying onto a local region of the body surface of the living body, may be used in combination with the mechanical wave(s) generated by the mechanical oscillating energy source to cause the microbubbles in the external type microbubble ultrasound contrast agent bursting to produce energy and shock waves. The energy and the shock waves from microbubble bursting cause minor and reversible damages on the contact area of the skin surface or mucous membrane, thereby increasing the percutaneous absorption of chemicals or small molecules. The microbubble ultrasound contrast agent may be widely used in medical or beauty fields, to help strengthen the absorption of painkillers after surgery or the absorption of beauty care ingredients.

The present invention provides an external type microbubble ultrasound contrast agent including a medium and a plurality of microbubbles dispersed in the medium. The medium is in a form of an aqueous solution or a gel form and a concentration of the microbubbles ranges from 4×10⁸ to 2×10¹⁰ particles/ml.

According to embodiments of the present invention, the material of the microbubbles is selected from albumin, polymers, liposomes, copolymers or mixtures thereof or a combination of thereof, and the medium is selected from an isotonic saline solution, an agar gel, an aloe gel, a topical gel or a combination of thereof.

According to embodiments of the present invention, the material of the microbubbles further includes hexose and/or pentose.

According to embodiments of the present invention, the hexose is dextrose.

According to embodiments of the present invention, the medium is a gel form medium and a content of the gel form medium is 0-0.2 percentages by weight of a total weight of the microbubble ultrasound contrast agent.

According to embodiments of the present invention, a particle size of the microbubbles ranges from 0.5 micrometers to 3.7 micrometers.

According to embodiments of the present invention, the microbubble ultrasound contrast agent further includes a chemical or small molecules, and the chemical or the small molecules are percutaneously absorbed by a biological body.

The present invention also provides a method for enhancing percutaneous absorption of a chemical or small molecules through a topical region of a biological body surface. A microbubble ultrasound contrast agent is applied to the topical region of the biological body surface. The microbubble ultrasound contrast agent comprises a medium and a plurality of microbubbles dispersed in the medium, the medium is in a form of an aqueous solution or a gel form, and a material of the microbubbles is selected from albumin, polymers, liposomes, copolymers or mixtures thereof or a combination of thereof. Also, a chemical or small molecules are applied to the topical region. Then, a mechanical oscillation wave source is applied to the topical region to be in direct contact with the topical region applied with the microbubble ultrasound contrast agent and the chemical or the small molecules. Through mechanical waves generated by the mechanical oscillating energy source acting on the microbubbles, the percutaneous absorption of the chemical or the small molecules is enhanced.

According to embodiments of the present invention, the material of the microbubbles further includes hexose and/or pentose.

According to embodiments of the present invention, the medium is an isotonic saline solution, and the hexose is dextrose.

According to embodiments of the present invention, a concentration of the microbubbles ranges from about 4×10⁸ to about 2×10¹⁰ particles/ml, relative to the total volume of the microbubble ultrasound contrast agent and the chemical or the small molecules.

According to embodiments of the present invention, using the chemical or the small molecules as a diluent, the microbubble ultrasound contrast agent is diluted 2-1000 times.

According to embodiments of the present invention, the steps of applying the microbubble ultrasound contrast agent and applying the chemical or the small molecules are performed individually and not at the same time.

According to embodiments of the present invention, a particle size of the microbubbles ranges from 0.5 micrometers to 3.7 micrometers.

According to embodiments of the present invention, the mechanical oscillation wave source includes an ultrasound source and/or a laser source.

The present invention also provides a method for preparing a microbubble ultrasound contrast agent. A microbubble material is mixed with a medium to form a mixed solution. An ultrasonic oscillating source is applied to oscillate the mixed solution for about 100 to about 140 seconds to form the microbubble ultrasound contrast agent including a plurality of microbubbles, wherein a particle size of the microbubbles ranges from 0.5 micrometers to 3.7 micrometers.

According to embodiments of the present invention, a concentration of the microbubbles ranges from 4×10⁸ to 2×10¹⁰ particles/ml.

According to embodiments of the present invention, the microbubble material is selected from albumin, polymers, liposomes, copolymers or mixtures thereof or a combination of thereof.

According to embodiments of the present invention, the microbubble material is albumin.

According to embodiments of the present invention, the medium is isotonic saline solution.

According to embodiments of the present invention, the microbubble material further includes pentose and/or hexose

According to embodiments of the present invention, the hexose is dextrose.

According to embodiments of the present invention, the microbubbles include octafluoropropane (C₃F₈) inside the microbubbles.

According to embodiments of the present invention, the microbubble material includes albumin and the medium is isotonic saline solution, the mixed solution includes albumin for from about 0.5 to about 1 wt %, and the formed microbubbles having average particle size for from about 0.5 to about 1 μm.

According to embodiments of the present invention, the microbubble material includes albumin or albumin and dextrose, the medium includes isotonic saline solution, the mixed solution includes albumin for from about 1 to about 1.5 wt %, or albumin for 1.32 wt % and the dextrose for from about 3 to about 7 wt %, or albumin for from about 0.5 to about 1 wt % and the dextrose for from about 8 to about 12 wt %, the formed microbubbles having average particle size for from about 1 to about 1.5 μm.

According to embodiments of the present invention, the microbubble material includes albumin and dextrose, the medium includes isotonic saline solution, the mixed solution includes albumin for 1.32 wt % and the dextrose for from about 8 to about 17 wt %, or albumin for from about 4.8 to about 5.2 wt % and the dextrose for from about 43 to about 47 wt %, the formed microbubbles having average particle size for from about 1.5 to about 2 μm.

According to embodiments of the present invention, the microbubble material includes albumin and dextrose, the medium includes isotonic saline solution, the mixed solution comprises albumin for 1.32 wt % and the dextrose for from about 18 to about 32 wt %, or albumin for from about 1.8 to about 2.2 wt % and the dextrose for from about 8 to about 12 wt %, or albumin for from about 3.3 to about 3.7 wt % and the dextrose for from about 43 to about 47 wt %, the formed microbubbles having average particle size for from about 2 to about 2.5 μm.

According to embodiments of the present invention, the microbubble material includes albumin or albumin and dextrose, the medium includes isotonic saline solution, the mixed solution includes albumin for from about 1.8 to about 5.2 wt %, albumin for 1.32 wt % and the dextrose for from about 38 to about 42 wt %, albumin for from about 3.3 to about 3.7 wt % and the dextrose for from about 8 to about 12 wt %, or albumin for from about 1.8 to about 2.2 wt % and the dextrose for from about 43 to about 47 wt %, the formed microbubbles having average particle size for from about 2.5 to about 3 μm.

According to embodiments of the present invention, the microbubble material includes albumin and dextrose, the medium includes isotonic saline solution, the mixed solution includes albumin for 1.32 wt % and the dextrose for from about 43 to about 47 wt %, or albumin for from about 4.8 to about 5.2 wt % and the dextrose for from about 8 to about 12 wt %, the formed microbubbles having average particle size for from about 3 to about 3.5 μm.

Based on the above, the present invention provides an external type ultrasound microbubble contrast agent(s), which can safely and effectively enhance the absorption or penetration of the chemical or small molecules at the topical region and avoid the risk of allergies by injecting the contrast agent into the body.

In order to make the aforementioned and other features and advantages of the disclosure comprehensible, several exemplary embodiments accompanied with figures are described in detail below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The accompanying drawings are included to provide a further understanding of the disclosure, and are incorporated in and constitute a part of this specification. The drawings illustrate embodiments of the disclosure and, together with the description, serve to explain the principles of the disclosure.

FIG. 1 is a flow chart illustrating the application of the ultrasound microbubble ultrasound contrast agent together with the treatment of ultrasound according to one embodiment of the present invention.

FIG. 2 is a schematic view of a penetration-through experimental system with the tissue simulator according to one embodiment of the present invention.

FIG. 3A shows the penetration depth of the agar stimulator in the penetration-through experiments according to one embodiment of the present invention.

FIG. 3B is a quantitative diagram showing the relationship of the penetration depth of the agar stimulator in the penetration-through experiments and the standing time according to one embodiment of the present invention.

FIG. 4A is a 100-fold magnification showing the percutaneous penetration depth of the penetration-through experiments.

FIG. 4B is a 400-fold magnification showing the percutaneous penetration depth of the penetration-through experiments.

FIG. 5 is a flow chart illustrating the preparation of the microbubble ultrasound contrast agent together with a genetransfer treatment of ultrasound according to one embodiment of the present invention.

FIG. 6 shows the results of the green fluorescent genetransfer efficiency with the microbubble contrast agent with different microbubble size.

FIG. 7 shows the fluorescent microscope images of the green fluorescent genetransfer experiments in FIG. 6.

FIG. 8 shows the penetration depth of the agar stimulator in the penetration-through experiments according to one embodiment of the present invention.

FIG. 9 is a quantitative diagram showing the relationship of the penetration depth of the agar stimulator in the penetration-through experiments according to one embodiment of the present invention.

FIG. 10 is a quantitative diagram showing the relationship of the percutaneous penetration depth of the pigskin in the penetration-through experiments according to one embodiment of the present invention.

FIGS. 11A-11C are 400-fold magnification showing the percutaneous penetration depth of the pigskin in the penetration-through experiments.

FIG. 12 shows the results of the delivery efficiency using different administration approaches of the microbubble contrast agent in the inner ear treatment experiments.

FIGS. 13A-13F show the delivery results of the green dye indicator entering into the round window membrane cells of the inner ear under different administration approaches.

FIGS. 14A-14B show the results of the auditory brainstem response tests of the animals following the animal tests.

### DETAILED DESCRIPTION OF DISCLOSED EMBODIMENTS

The microbubble ultrasound contrast agent of the present invention may be of an aqueous solution or a gel form and contain the microbubbles of a specific particle size and a specific concentration. According to the material of the microbubbles contained therein, the microbubble ultrasound contrast agent can be divided roughly into three categories: albumin microbubbles, liposome microbubbles or polymer microbubbles. The microbubbles contained in the microbubble ultrasound contrast agent have stable shells and may be used to enhance the scattering signals of reflected ultrasound. Under various ultrasound energy intensities, using the microbubble ultrasound contrast agent can increase the penetration depth (i.e. absorption efficiency) and/or the amount of penetration (i.e. absorption) of the chemicals or small molecules at the applied area.

Taking the lipid microbubble ultrasound contrast agent as an example, under the action of very low sound field energy of the mechanical index (MI) less than 0.05-0.1, the microbubble ultrasound contrast agent oscillate linearly and symmetrically. When the mechanical index is raised to 0.1-0.3, the microbubble ultrasound contrast agent is being squeezed more than being relaxed. At this time, although the microbubble ultrasound contrast agent does not have considerable cavitation, the microbubble ultrasound contrast agent has significant nonlinear response and the signal spectrum has obvious harmonic components. Harmonic imaging can effectively increase the scattering ratios of the bubbles to the tissues. However, in the case of high sound pressure (mechanical index greater than 0.3-0.6), the microbubble ultrasound contrast agent may endure big squeezes and relaxations, leading to the bursting of the microbubbles in the microbubble ultrasound contrast agent into pieces and then linear scattering and cavitation. Shock waves generated by cavitation can cause membrane perturbation and increase its permeability. According to the studies, under the high sound field, cavitation of the microbubble ultrasound contrast agent is used to augment microvascular leakage, inflammatory cell infiltrations, hemolysis or even capillary ruptures and so on.

The present invention provides an external type microbubble ultrasound contrast agent, and the microbubble ultrasound contrast agent may be applied to a topical region of the body surface of the living body (i.e. external) by coating, painting or spraying. The external use microbubble ultrasound contrast agent(s) can enhance the absorption efficacy of chemicals or small molecules that are mixed with the microbubble ultrasound contrast agent(s) by the topical region of the living body. Compared to the previously used microbubble ultrasound contrast agent that is injected into the living body's circulatory systems, the microbubble ultrasound contrast agent of the present invention is designed to be the medium disposed between the ultrasound probe and the action site (a local region of the biological body surface, such as, face, ear cavity or joints, etc.). That is, the microbubbles exist stably in the microbubble ultrasound contrast agent and the microbubbles are in direct contact with the ultrasound probe to induce cavitation under the ultrasound energy, thereby strengthening the absorption and utilization of chemicals or small molecules applied to shallow parts of the body surface. Further, since the chemicals or small molecules mixed with the microbubble ultrasound contrast agents of the present invention are not enveloped within the microbubbles, the chemicals or small molecules may be used with the microbubble ultrasound contrast agents of the present invention separately or in combination. In other words, these chemicals, small molecules may be applied or coated to the outside surface of the living body in different orders.

The microbubble ultrasound contrast agents of the present invention may be designed to adjust the microbubble concentration and/or medium tension to make the formulation of the microbubble ultrasound contrast agent appropriate for being in direct contact with the ultrasound probe. The medium of the microbubble ultrasound contrast agents may be an aqueous medium or in a gel form, and the medium still has effective acoustic transfer properties with a specific concentration of microbubbles. The material of the microbubbles in the microbubble ultrasound contrast agent may be albumin, liposomes, polymers, copolymers or mixtures of the foregoing material(s), or a combination of the above. The aqueous medium may be isotonic saline solution. In some embodiments, the material of the microbubbles may further include hexose or pentose.

The present invention also provides a method for preparing a microbubble ultrasound contrast agent. A microbubble material is mixed with a medium to form a mixed solution. An ultrasonic oscillating source is applied to oscillate the mixed solution for about 100 to about 140 seconds to form the microbubble ultrasound contrast agent including a plurality of microbubbles, wherein a particle size of the microbubbles ranges from 0.5 micrometers to 3.7 micrometers. The particle size of the microbubbles ranges may be adjusted by using different ratio of the microbubble material and the medium. The material of the microbubbles in the microbubble ultrasound contrast agent may be albumin, liposomes, polymers, copolymers or mixtures of the foregoing material(s), or a combination of the above. The aqueous medium may be isotonic saline solution. In some embodiments, the material of the microbubbles may further include hexose or pentose.

The present invention also describes the application of the ultrasound microbubble composition for external uses, applied to a local region of the body surface to promote the penetration efficacy of chemicals or small molecules through the skin or mucous membranes of the local region, so as to strengthen the absorption of those chemicals or small molecules. Such external use ultrasound microbubble composition includes at least one medium and a plurality of microbubbles dispersed in the medium. The medium may be in the form of an aqueous solution or a colloid suspension, and the material of the microbubbles may be selected from albumin, polymers, liposomes, copolymers or mixtures of the aforementioned material(s), or a combination of the above. The aqueous medium may be an isotonic saline solution. In some embodiments, the material of the microbubbles may further include hexose or pentose. When used, the topical ultrasound microbubble composition may be diluted 2-1000 times, from the original prior concentration of 4×10⁸-2×10¹⁰ particle/ml to the concentration range of 4×10⁵-1×10¹⁰ particles/ml by adding the medium. In some embodiments, the topical ultrasound microbubble concentration may be 4×10⁸-4×10⁹ particle/ml and dilute to the concentration range of 4×10⁵-2×10⁹ particles/ml by adding the medium. In some embodiments, the microbubble ultrasound contrast agent may be diluted to the concentration range of 2×10⁶-2×10⁸ particles/ml.

The following examples are based on albumin microbubble ultrasound contrast agent(s), for example, but the microbubble ultrasound contrast agent of the present invention is not limited to the content of the following Examples.

### EXAMPLES

### Preparation Steps of Topical Microbubble Contrast Agent

Method one: the preparation of aqueous microbubble ultrasound contrast agent(s).

The isotonic saline solution and 1.2 wt % of human serum albumin (HSA, purchased from Octapharma, Vienna, Austria) were uniformly mixed into 10 ml of the solution, filled with C₃F₈ gas, and oscillated for two minutes using the ultrasonic cell processor to prepare the microbubble ultrasound contrast agent. The microbubble ultrasound contrast agent contains microbubbles, formed in the oscillation process, with C₃F₈ gas sealed by albumin shells. After the oscillation was complete, the microbubble ultrasound contrast agent was dispensed into microcentrifuge tubes, placed in micro-centrifuge for separation (speed: 1200 rpm (128.7 g), time: 2 minutes), extract subnatant and add the appropriate amount of saline for storage at 4° C. refrigerator. For the contrast agent(s) used in this experiment, a concentration of the microbubbles is about 2×10⁹ particles/ml and the particle size distribution of the microbubbles is about 0.5˜3.7 μm.

Component A: using the isotonic saline solution as the medium to adjust concentrations of the microbubbles for the various commercial lipid-shell microbubbles (including phospholipid microbubbles SonoVue® (purchased from Bracco Diagnostics, Milan, Italy), Definity (purchased from Lantheus Medical Imaging Inc, Billerica, Mass.), or Targestar (purchased from Targeson, La Jolla, Calif.)) or the prepared albumin-shell microbubble ultrasound contrast agent as mentioned above, to the concentrations of 1×10⁹-2×10⁹ particles/ml (the microbubble liquid).

Component B: chemical, biological and other small molecules or drugs to be used together are prepared. The substance to be used together should be formulated in the desired state of an aqueous solution, an emulsion or a gel, so that the substance is isotonic with human cells with a pH=7.4. For example, chemical, biological or small molecule drugs may be painkillers (such as diclofenac), arbutin, vitamin C phosphate magnesium salt, whitening ingredients (such as nonapeptide-1), gentamycin or glucocorticoid and other applicable substances.

Component C: Component B is used a diluent to dilute Component A 2-1000 times and the composition obtained after dilution is applied to the surface of the body. Most preferably, Component B is used to dilute Component A 2-40 times; more preferably, Component B is used to dilute Component A 30-150 times. Also, Component B is used to dilute Component A 100-1000 times. According to experimental results, the 10-fold dilution was the best dilution solution applied to the skin surface. Other dilution ratios are effective, and the dilution ratios should be adjusted depending on the application region. In general, in the topical microbubble contrast agent, the concentration of microbubbles ranges may be about 4×10⁵-1×10¹⁰ particles/ml, preferably from about 2×10⁶-2×10⁸ particles/ml.

In general, the ultrasonic probe directly applied on the outer surface of the living body is in direct contact with Component C for local application of ultrasound with the power of 0.1-5 W/cm² and the mechanical index (MI)<1.9. In addition, the ultrasonic energy applied with Component C may be replaced by other sources capable of generating the mechanical oscillation energy or may be used in combination with other devices. For example, the therapeutic laser beams may be applied to the local region with Component C. The mechanical oscillation means functioned with Component C and the corresponding replacements may be easily conceived by the skilled persons, and examples herein are not used to limit the applied energy sources.

Method Two: the preparation of colloidal microbubble ultrasound contrast agent(s):

The isotonic saline solution is used to prepare 0.2 wt % or less of the agar gel, aloe vera gel, or other topical gel.

Component D: The topical gel as described above is used as the medium, and the microbubble ultrasound contrast agent and 0.2 wt % or less (for example, 0.1 wt % or 0.15 wt %) of the agar gel, aloe (vera) gel, or other topical gel were mixed and the microbubble concentration was adjusted to approximately 1×10⁹-2×10⁹ particles/ml (the microbubble liquid).

Component E: chemical, biological and other small molecules or drugs to be used together are prepared. The substance to be used together should be formulated in the desired state of an aqueous solution, an emulsion or a gel, so that the substance is isotonic with human cells with a pH=7.4. For example, chemical, biological or small molecule drugs may be painkillers (such as diclofenac), arbutin, vitamin C phosphate magnesium salt, whitening ingredients (such as nonapeptide-1), gentamycin or glucocorticoid and other applicable substances.

Component F: Component E is used a diluent to dilute Component D 2˜1000 times and the composition obtained after dilution is applied to the surface of the body. Most preferably, Component E is used to dilute Component D 2-40 times; more preferably, Component E is used to dilute Component D 30-150 times. Also, Component E is used to dilute Component D 100-1000 times. According to experimental results, the 10-fold dilution was the best dilution solution applied to the skin surface. Other dilution ratios are effective, and the dilution ratios should be adjusted depending on the application region. In general, in the topical microbubble contrast agent, the concentration of microbubbles ranges may be about 4×10⁵-1×10¹⁰ particles/ml, preferably from about 2×10⁶ to 2×10⁸ particles/ml.

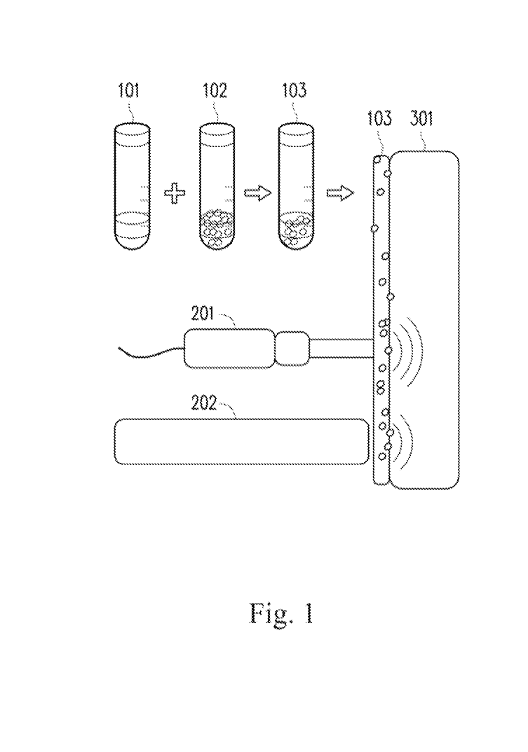

In general, the ultrasonic probe directly applied on the outer surface of the living body is in direct contact with Component F for local application of ultrasound with the power of 0.1-5 W/cm² and the mechanical index (MI)<1.9. In addition, the therapeutic laser beams may be applied to the local region with Component F. To illustrate the principle and design of the present invention, the following embodiments are provided for descriptions. FIG. 1 is a flow chart illustrating the application of the ultrasound microbubble ultrasound contrast agent together with the treatment of ultrasound according to one embodiment of the present invention. First, Component A or Component D 101 is fully mixed with Component B or Component E 102 to obtain Component C or Component F 103, and the resultant Component C or Component F 103 is evenly spread onto the surface of the local region 301. Then the ultrasound probe 201 directly contacts the Component C or Component F 103 spread on the surface of the local region 301, and applying ultrasound (represented by arc lines) in order to enhance the penetration and absorption of the above components or chemicals. The system may further include air gun or laser device 202. The ultrasonic signals of the aqueous or colloid (gel) microbubble ultrasound contrast agent 103, compared with that of water, are pretty significant and have the fundamental frequency and harmonic signals, which keeps various physical effects induced by the ultrasound.

Percutaneous Penetration Experiments

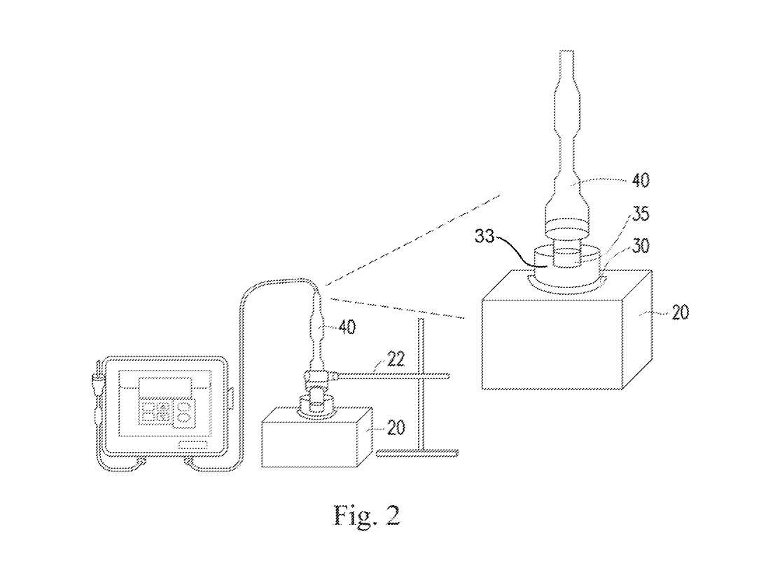

FIG. 2 is a schematic view of a penetration-through experimental system with the tissue simulator according to one embodiment of the present invention. At first, a skin tissue simulator 20 formed of 0.3 wt % agarose gel (agar gel), which simulates the human skin tissue(s), is provided for conducting the penetration-through experiments. The mechanical oscillation wave source may be the ultrasound probe. The ultrasound probe) 40 is mounted on the dropper rack 22 and the ultrasound probe 40 is set at a distance of about 5 mm from the tissue stimulator 20. The conductive gel 35 is disposed on the probe 40 so that the conductive gel 35 is located apart from the tissue stimulator 20 with a distance of about 3 mm. See FIG. 2, the perfusion zone 33 is placed above the tissue stimulator 20 and the conductive gel 30 is located outside of the perfusion zone 33. The gel-based microbubble ultrasound contrast agent of this invention is used as the conductive gel 35, and the small molecules or chemicals may be placed in the perfusion zone 33. In some embodiments, the conductive gel 30 may spread on the tissue surface to seal the perfusion zone 33, preventing the leakage of the chemicals in the perfusion zone 33. In some embodiments, the conductive gel 35 may be omitted when the chemicals itself in the perfusion zone 33 can conduct ultrasonic. The microbubble ultrasound contrast agent may mix with chemicals or the small molecules and oscillating by ultrasound at once, or use the ultrasound to oscillate the microbubble ultrasound contrast agent to damage the skin surface first, then use the ultrasound to oscillate the chemicals or the small molecules to enhance the delivery into a body.

Ultrasound applications process: The conductive gel was coated and the ultrasound was applied for 1 minute. The surface of the tissue stimulator is rinsed three times (1000 μl). The control group utilized the saline solution of 0.01 wt % Evans blue dye (0.0001 g Evans blue dye dissolved in 1 ml saline). After the application of the ultrasound, the tissue stimulator was placed in the perfusion zone for 2 to 30 minutes (for example: 5 minutes, 10 minutes, 15 minutes or 20 minutes). After placing in the perfusion zone for a predetermined time (the standing time), the penetration depth of the dye (dye penetration depth) of the tissue stimulator was observed by the microscope and the results were processed by MATLAB program to calculate the dye penetration depth.

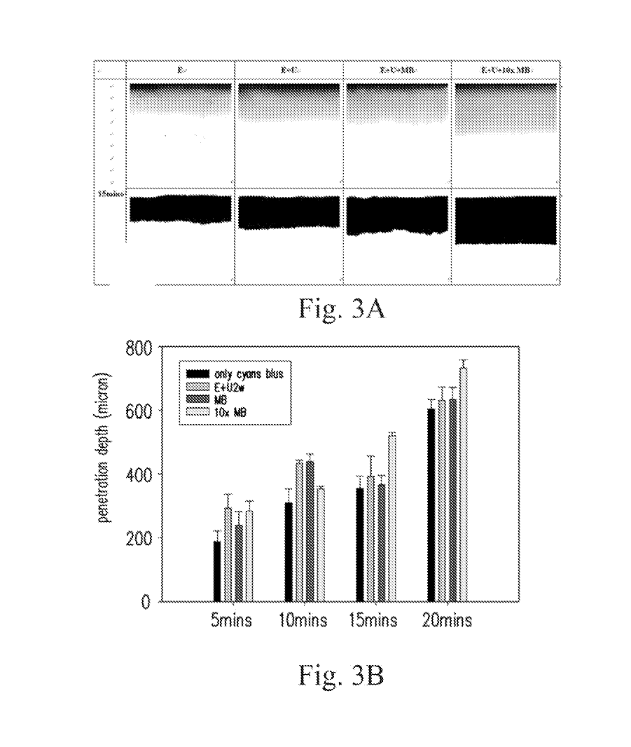

In the following three experiments, different parameters were changed to find the best conditions for the penetration depth of the dye. (1) only Evans blue dye (represented by E); (2) Evan blue dye+ultrasound (represented by E+U); (3) Evans blue dye+ultrasound+microbubble contrast agent (represented by E+U+MB or MB); (4) Evans blue dye+ultrasound+10-fold dilution of microbubble contrast agent (represented by E+U+10×MB or 10×MB); E meant for Evans blue dye; U meant for ultrasound; MB meant for microbubble contrast agents; 10×MB meant for 10-fold dilution of microbubble contrast agent. After the application of the ultrasound and placing in the perfusion zone for a predetermined time, the dye penetration depth was observed by the microscope and the results were processed by MATLAB program to calculate the dye penetration depth. FIG. 3A shows the penetration depth of the agar stimulator in the penetration-through experiments according to one embodiment of the present invention. FIG. 3B is a quantitative diagram showing the relationship of the penetration depth of the agar stimulator in the penetration-through experiments and the standing time according to one embodiment of the present invention.

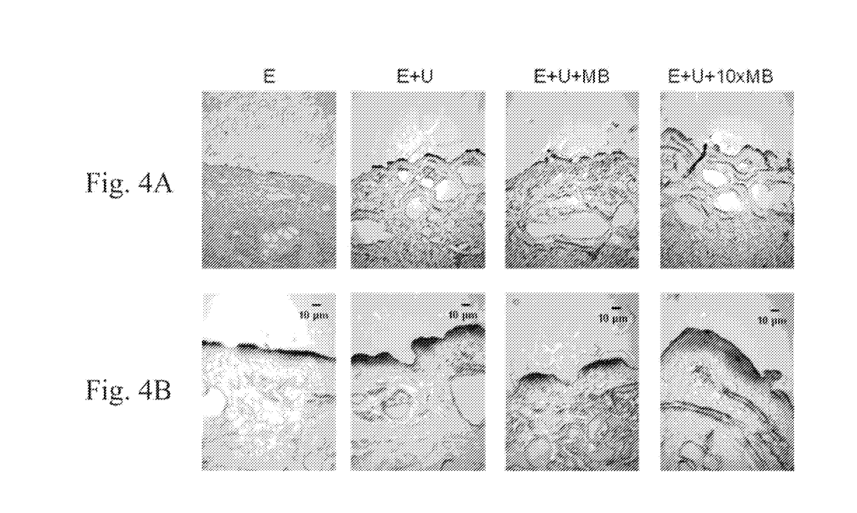

In another experiment, the perfusion zone was placed on the pigskin of 2 mm thickness for conducting the percutaneous penetration experiments, and the experimental system and the methods were similar to the penetration-through experiments of the agar stimulator. The results of the penetration-through experiments are shown in FIGS. 4A-4B. FIG. 4A is a 100-fold magnification showing the percutaneous penetration depth of the penetration-through experiments, while FIG. 4B is a 400-fold magnification showing the percutaneous penetration depth of the penetration-through experiments.

From the experimental results of the penetration-through experiments, the microbubble ultrasound contrast agent of this invention used in combination with the ultrasound can make the dye penetrate deeper or more uniformly. With respect to the agar stimulator, the penetration-through experiments conducted on the pigskin penetration experiments proves that the microbubble ultrasound contrast agent of the present invention do enhance the penetration of small molecules (permeation). During application, it is better to dilute the external use microbubble ultrasound contrast agent of the present invention with a diluent at the dilution ratio of about 1:2 dilution to 1:1000 dilution. The diluent may be the medium itself contained in the microbubble contrast agent of the present invention to increase the proportion of the medium; or the diluent may be a small molecule, a chemical or a medicinal ingredient itself. Further, the medium of the external use microbubble contrast agent is not limited to the traditional liquid state isotonic medium. The microbubbles in the microbubble contrast agent may be made of albumin, polymers, liposomes, copolymers, mixtures or a combination of the aforementioned materials, for example. The microbubble ultrasound contrast agent for topical uses may include the microbubbles in the concentration range of 4×10⁵-1×10¹⁰ particles/ml, preferably about 2×10⁶-2×10⁸ particles/ml. If a gel medium is used, relatively to the total weight of the composition of the microbubble contrast agent and the medium, the content of the gel medium may be less than or equivalent to 0.2 wt %, which can effectively transfer sound waves. Alternatively, an isotonic saline solution may be used as the medium.

Penetration experiment for microbubble ultrasound contrast agent with different particle sizes

Preparation steps of topical microbubble contrast agent with different particle sizes:

The preparation method is similar with the above-mentioned method one to prepare the microbubble ultrasound contrast agent. The preparation method includes mixing a microbubble material and a medium to form a mixed solution, and applying an ultrasonic oscillating source oscillating the mixed solution for about 100 to about 140 seconds to form the microbubble ultrasound contrast agent including a plurality of microbubbles. The panicle size of the microbubbles ranges from 0.5 micrometers to 3.7 micrometers. The medium may be isotonic saline solution. And the microbubble material is serum albumin. The microbubble material may also include pentose or hexose, such as dextrose. Using specific ratio of the concentration of the serum albumin, isotonic saline solution and dextrose may prepare microbubble ultrasound contrast agent with specific particle sizes.

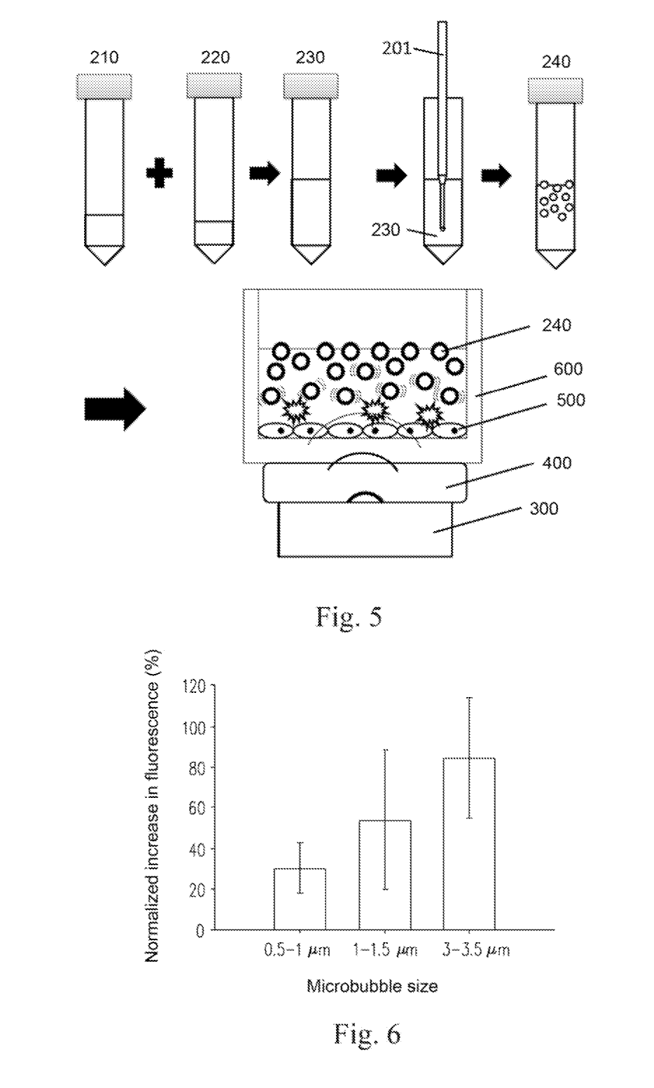

Referring to FIG. 5, FIG. 5 is a flow chart illustrating the preparation of the ultrasound microbubble ultrasound contrast agent together with a genetransfer treatment of ultrasound according to one embodiment of the present invention. The microbubble material 210 is mixed with the medium 220 to form the mixed solution 230. The mixed solution 230 is oscillated by an ultrasonic oscillating source 201, such as an ultrasonic cell processor, with 20 kHz for about 100 to about 140 seconds to form the microbubble ultrasound contrast agent 240 which includes a plurality of microbubbles. The particle size of the microbubbles ranges from 0.5 micrometers to 3.7 micrometers, and the concentration of the microbubbles ranges from 4×10⁸ to 2×10¹⁰ particles/ml, and in some embodiments, from 4×10⁸ to 4×10⁹ particles/ml. The different ratio of the medium and the microbubble material may form microbubble ultrasound contrast agent having microbubbles with different size by the same process. The medium may be isotonic saline solution and the microbubble material may be albumin or albumin and dextrose. The ratio of the medium and the microbubble material and the particle size of the microbubbles may refer to the following table 1, in which the concentrations are all weight percentage.

TABLE 1

| | | | Albumin |
|---|---|---|---|
| Microbubble | Albumin | Dextrose | concentration |
| particle | concentration | concentration | (wt %)/dextrose |
| size | (wt %) | (wt %) | concentration |
| (μm) | (0% dextrose) | (1.32% albumin) | (wt %) |
| <1 | 0.5-1 | N/A | N/A |
| 1-1.5 | 1-1.5 | 4-6 | 0.5-1/8-12 |
| 1.5-2 | N/A | 8-17 | 4.8-5.2/43-47 |
| 2-2.5 | N/A | 18-32 | 1.8-2.2/8-12, |
| | | | 3.3-3.7/43-47, |
| 2.5-3 | 1.8-5.2 | 38-42 | 3.3-3.7/8-12, |
| | | | 1.8-2.2/43-47 |
| 3-3.5 | N/A | 43-47 | 4.8-5.2/8-12 |

Table 1 shows the relationship of concentration of the albumin and the dextrose in the mixed solution, which the medium is isotonic saline solution, and the microbubble concentration according to embodiments of the present invention.

As shown in Table 1, the mixed solution includes albumin for from about 0.5 to about 1 wt %, the formed microbubbles having average particle size for from about 0.5 to about 1 μm. The mixed solution includes albumin for from about 1 to about 1.5 wt %, or albumin for 1.32 wt % and the dextrose for from about 3 to about 7 wt %, or albumin for from about 0.5 to about 1 wt % and the dextrose for from about 8 to about 12 wt %, the formed microbubbles having average particle size for from about 1 to about 1.5 μm. The mixed solution includes albumin for 1.32 wt % and the dextrose for from about 8 to about 17 wt %, or albumin for from about 4.8 to about 5.2 wt % and the dextrose for from about 43 to about 47 wt %, the formed microbubbles having average particle size for from about 1.5 to about 2 μm. The mixed solution includes albumin for 1.32 wt % and the dextrose for from about 18 to about 32 wt %, or albumin for from about 1.8 to about 2.2 wt % and the dextrose for from about 8 to about 12 wt %, or albumin for from about 3.3 to about 3.7 wt % and the dextrose for from about 43 to about 47 wt %, the formed microbubbles having average particle size for from about 2 to about 2.5 μm. The mixed solution includes albumin for from about 1.8 to about 5.2 wt %, albumin for 1.32 wt % and the dextrose for from about 38 to about 42 wt %, albumin for from about 3.3 to about 3.7 wt % and the dextrose for from about 8 to about 12 wt %, or albumin for from about 1.8 to about 2.2 wt % and the dextrose for from about 43 to about 47 w %, the formed microbubbles having average particle size for from about 2.5 to about 3 μm. The mixed solution includes albumin for 1.32 wt % and the dextrose for from about 43 to about 47 wt %, or albumin for from about 4.8 to about 5.2 wt % and the dextrose for from about 8 to about 12 wt %, the formed microbubbles having average particle size for from about 3 to about 3.5 μm. Therefore, using the specific concentration of the albumin and dextrose followed by the ultrasonic oscillation for about 100 to about 140 seconds may form the microbubble ultrasound contrast agent with microbubbles having specific particle size. For the microbubble ultrasound contrast agent(s) used in this experiment, a concentration of the microbubbles is about 2×10⁹ particles/ml. In some embodiments, the formed microbubble ultrasound contrast agent has a concentration of the microbubbles from about 4×10⁸ to about 2×10¹⁰ particles/ml. In some embodiments, the microbubble concentration is from about 4×10⁸ to 4×10⁹ particles/ml. The microbubble ultrasound contrast agent may storage at 4° C. refrigerator. In some embodiments, the oscillating step may fill with C₃F₈ gas to form the microbubbles having C₃F₈ inside, which may use in human body. The microbubble ultrasound contrast agent may be diluted or directly used on the topical region and be oscillated to help to overcome the skin barrier.

In order to know the effect of the microbubble size of the microbubble ultrasound contrast agent for the percutaneous absorption of the chemicals, a genetransfer experiment and a percutaneous penetration experiments are done.

Genetransfer Experiment

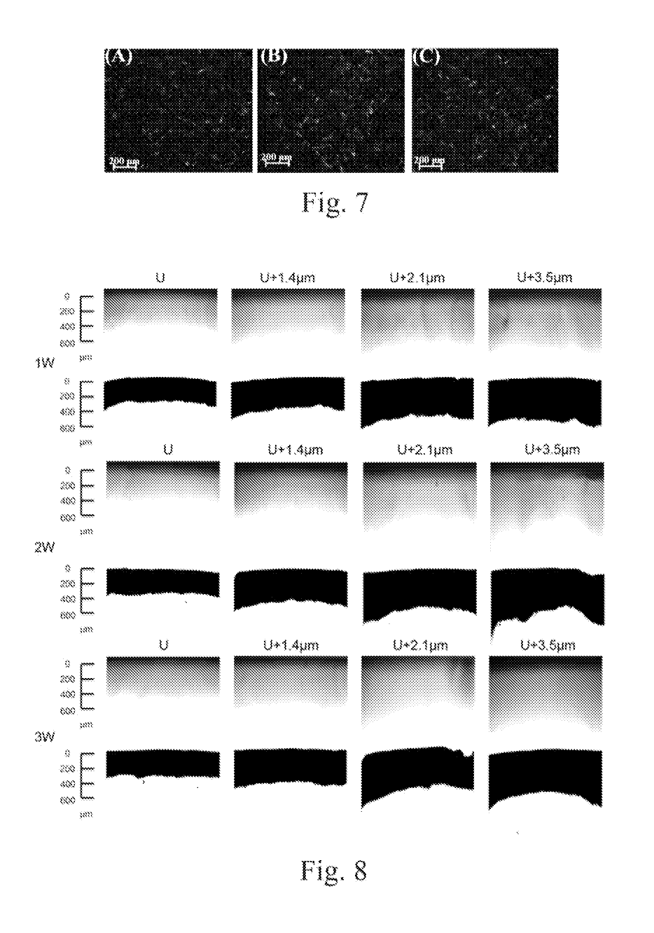

Referring to FIG. 5, the experiment use HEI-OCL cell to operate a green fluorescent genetransfer. A layer of cell 500 is cultured in a petri dish 600, and the formed microbubble ultrasound contrast agent 240 is poured into the petri dish 600, making the microbubble ultrasound contrast agent 240 contacts with the cell 500. A mechanical oscillation wave source 300, which may be an ultrasound probe, contacts and oscillates a bottom of the petri dish 600. And a conductive gell is spreaded between the bottom of the petri dish 600 and the mechanical oscillation wave source 300 to enhance the energy conductivity. An ultrasonic energy is emitted to oscillate the microbubble ultrasound contrast agent 240 to damage the cell surface and enhance the cell permeability, in which ultrasonic power is 0.1-5 W/cm². Than the microbubble ultrasound contrast agent 240 is removed, and the surface of the cell 500 is washed. A liposome encapsulating plasmid which has a green fluorescent gene puts into the petri dish 600 and contacts the cell 500 to perform the genetransfer. And the green fluorescent genetransfer efficiency is observed after 24 hours. Using three groups of the microbubble ultrasound contrast agent 240 with microbubble diameter in a range of 0.5-1 μm, 1-1.5 μm, and 3-3.5 μm to do the genetransfer experiment. The experiment results are shown in FIGS. 6 and 7.

Referring to FIG. 6, FIG. 6 shows the results of the green fluorescent genetransfer efficiency with the microbubble contrast agent with different microbubble sizes. The FIG. 6 shows that the larger the microbubble diameter is, the higher the green fluorescent genetransfer efficiency is. Referring to FIG. 7, FIG. 7 shows the fluorescent microscope images of the green fluorescent genetransfer experiments in FIG. 6. The image A refers to the microbubble diameter for 0.5-1 μm, the image B refers to the microbubble diameter for 1-1.5 μm, and the image C refers to the microbubble diameter for 3-3.5 μm. As shown in FIG. 7, the image C is brighter than image B and A, showing that the larger microbubble diameter makes higher genetransfer efficiency under the same ultrasonic energy. Thus the microbubble contrast agent with different microbubble diameter may be chose for different genetransfer demand.

Percutaneous Penetration Experiments with Different Microbubble Sizes

The percutaneous penetration experiments with different microbubble diameters are also been made. Referring to FIG. 2, the experiment process is the same with the embodiments in FIG. 2. A skin tissue simulator 20 is formed of 0.3 wt % agarose gel (agar gel). The mechanical oscillation wave source may be an ultrasound probe 40. The ultrasound probe 40 is mounted on the dropper rack 22 and the ultrasound probe 40 is set at a distance of about 5 mm from the tissue stimulator 20. The conductive gel 35 is disposed on the probe 40 so that the conductive gel 35 is located apart from the tissue stimulator 20 with a distance of about 3 mm. The perfusion zone 33 is placed above the tissue stimulator 20 and the conductive gel 30 is located outside of the perfusion zone 33.

The microbubble ultrasound contrast agent is formed according to the above-mentioned methods and table 1. Three groups of the microbubble ultrasound contrast agent are formed with microbubble size are 1.4 μm, 2.1 μm, and 3.5 μm. The microbubble ultrasound contrast agents are diluted for 10 times by adding the isotonic saline solution, the microbubble concentration are diluted to 2×10⁸ particles/ml. And the dye for observing the penetration depth in the skin tissue simulator is Evans blue which mixed with water to form a 0.25 wt % dye solution.

Ultrasound applications process: The microbubble ultrasound contrast agent was spreaded on the skin tissue simulator surface and the ultrasound was applied for one minute. The surface of the tissue stimulator is rinsed three times (1000 μl). The control group utilized the saline solution of 0.25 wt % Evans blue dye. After the application of the ultrasound with the microbubble ultrasound contrast agent, the 0.25 wt % Evans blue dye solution was placed in the perfusion zone and the ultrasound was applied for one minute to force the dye diffused into the tissue simulator. After the dye placed in the perfusion zone for a predetermined time, the penetration depth of the dye of the tissue stimulator was observed by the microscope and the results were processed by MATLAB program to calculate the dye penetration depth.

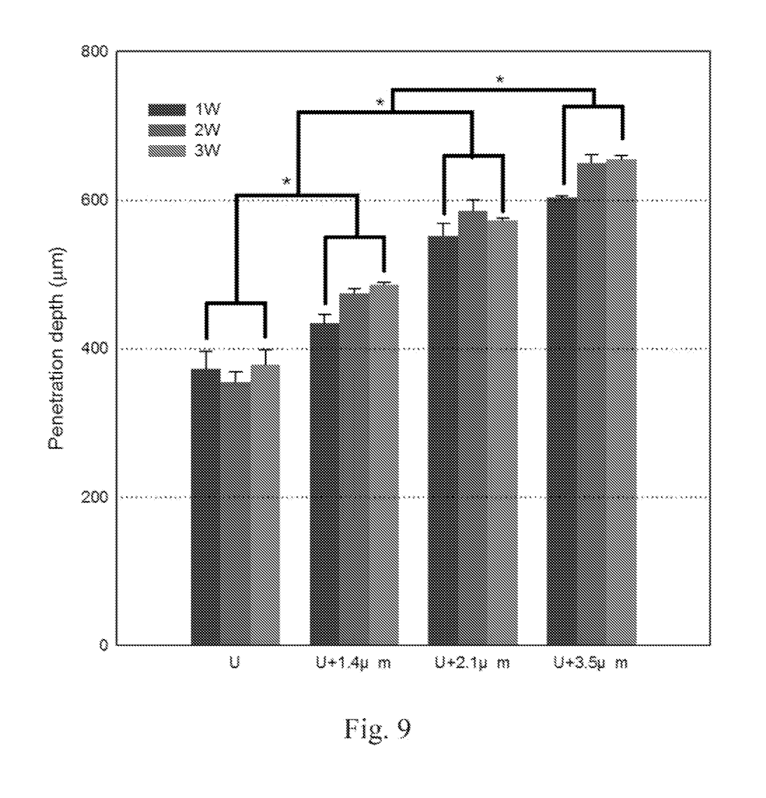

In the following experiments, different parameters were changed to find the best conditions for the penetration depth of the dye. (1) only Evans blue dye (represented by U); (2) Evan blue dye+microbubble ultrasound contrast agent with 1.4 μm microbubble size (represented by U+1.4 μm); (3) Evans blue dye+microbubble ultrasound contrast agent with 2.1 μm microbubble size (represented by U+2.1 μm); (4) Evans blue dye+microbubble ultrasound contrast agent with 3.5 μm microbubble diameter (represented by U+3.5 μm). And each group further had three experiments with the different ultrasound energy for 1 W/cm², 2 W/cm², and 3 W/cm². The ultrasound applied for 1 minute. FIG. 8 shows the penetration depth of the agar stimulator in the penetration-through experiments according to one embodiment of the present invention. And FIG. 9 is a quantitative diagram showing the relationship of the penetration depth of the agar stimulator in the penetration-through experiments according to one embodiment of the present invention. FIGS. 8 and 9 shows that the microbubble ultrasound contrast agent with larger microbubble diameter may provide better penetration depth for the dye solution. The penetration depth result for each experiment group is listed in table 2. The penetration depth for the dye of the control group is 249±20 μm.

TABLE 2

| | 1 W | 2 W | 3 W |
|---|---|---|---|
| U only | 373 ± 23 | 354 ± 14 | 379 ± 20 |
| U + 1.4 μm | 434 ± 12 | 475 ± 6 | 487 ± 3 |
| U + 2.1 μm | 551 ± 17 | 586 ± 14 | 573 ± 3 |
| U + 3.5 μm | 603 ± 2 | 650 ± 12 | 655 ± 6 |

In another experiment, the skin tissue simulator formed form the agar gel is replaced by a pigskin of 2 mm thickness. The perfusion zone was placed on the pigskin for conducting the percutaneous penetration experiments, and the experimental system and the methods were similar to the penetration-through experiments of the agar stimulator. The experiment also use different ultrasound energy for 1 W/cm², 2 W/cm² and 3 W/cm², and the particle sizes of the microbubbles in the microbubble ultrasonic contrast agent are about 1.4 μm, 2.1 μm, and 3.5 μm. The microbubble ultrasound contrast agent is applied on the pigskin, and the ultrasonic energy is applied to oscillate the skin for 1 minute to damage the cell on the skin surface. Then the microbubble ultrasonic contrast agent was washed away. The dye solution was placed in the perfusion zone and the ultrasound was applied for 15 minutes to force the dye diffused into the tissue simulator.

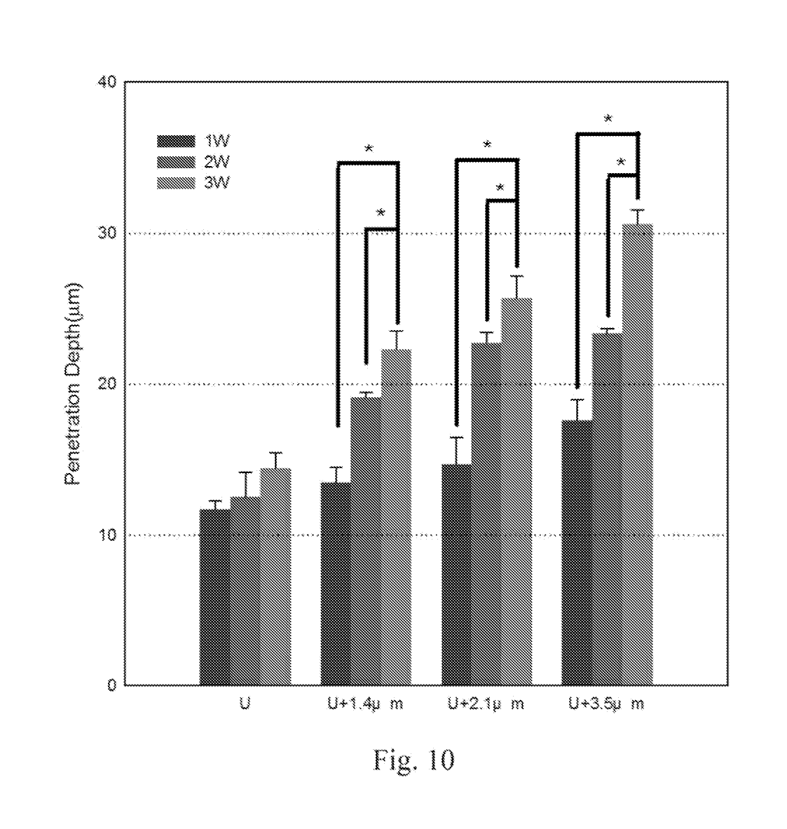

The results of the penetration-through experiments are shown in FIGS. 10 and 11A-11C. FIG. 10 is a quantitative diagram showing the relationship of the percutaneous penetration depth of the dye in the penetration-through experiments according to one embodiment of the present invention. FIGS. 11A-11C are 400-fold magnification showing the percutaneous penetration depth of the dye in the penetration-through experiments. FIG. 11A is the results of using ultrasonic energy with 1 W/cm² to enhance the penetration depth of the dye. FIG. 11B is the results of using ultrasonic energy with 2 W/cm² to enhance the penetration depth of the dye. And FIG. 11C is the results of using ultrasonic energy with 3 W/cm² to enhance the penetration depth of the dye. The experiment result shows that the larger the microbubble size is, the deeper the penetration depth of the dye; and the larger the ultrasonic energy is, the deeper the penetration depth of the dye. For the experiment group of applying ultrasonic energy of 1 W/cm², even the result for the penetration depth of each group with different microbubble sizes are different, the difference is not apparent. But in the experiment groups for ultrasonic energy with 2 and 3 W/cm², the penetration depth of the dye in different microbubble size group have larger difference. The experiment result is the same with the result using the skin tissue simulator formed from the agar gel. The penetration depth result for each experiment group is listed in table 3. The average penetration depth of the dye for the control group is 12.24 μm.

TABLE 3

| | 1 W | 2 W | 3 W |
|---|---|---|---|
| U Only | 12.78 | 14.17 | 15.5 |
| U + 1.4 μm | 14.6 | 18.79 | 22.12 |
| U + 2.1 μm | 16 | 22.55 | 27.28 |
| U + 3.5 μm | 19 | 25.02 | 30.61 |

From the experimental results of the penetration-through experiments, the microbubble ultrasound contrast agent of this invention used in combination with the ultrasound can make the dye penetrate deeper or more uniformly. With respect to the agar stimulator, the penetration-through experiments conducted on the pigskin penetration experiments proves that the microbubble ultrasound contrast agent of the present invention do enhance the penetration of small molecules (permeation). Also by the experiment results listed in table 2 and 3, microbubble ultrasound contrast agent with different microbubble size may be chosen to reach the specific penetration depth of the dye or chemicals. And microbubble ultrasound contrast agent with specific microbubble size is easily to be formed with the specific ratio of albumin, dextrose and isotonic saline solution according to table 1.

During application, it is better to dilute the external use microbubble ultrasound contrast agent of the present invention with a diluent at the dilution ratio of about 1:2 dilution to 1:1000 dilution. The diluent may be the medium itself contained in the microbubble contrast agent of the present invention to increase the proportion of the medium; or the diluent may be a small molecule, a chemical or a medicinal ingredient itself. Further, the medium of the external use microbubble contrast agent is not limited to the traditional liquid state isotonic medium. The microbubbles in the microbubble contrast agent may be made of albumin, polymers, liposomes, copolymers, mixtures or a combination of the aforementioned materials, for example. The microbubble ultrasound contrast agent for topical uses may include the microbubbles in the concentration range of 4×10⁵-1×10¹⁰ particles/ml, preferably 2×10⁶-2×10⁸ particles/ml. Alternatively, an isotonic saline solution may be used as the medium.

For medical applications, the external use microbubble contrast agent of the present invention may be used in the ear treatments. The microbubble contrast agent of this invention is mixed with the dye and/or one or more medical ingredients and administrated to the inner ear of guinea pigs. The administration of the mixtures may be conducted in different ways to test the delivery efficiency of the dye or the ingredient.

Animal Test Procedures

The animals used in the test are 60 guinea pigs with the normal Preyer's reflex to the sound(s) and are divided into three groups with the following experimental conditions: (1) the tympanic bullae of 24 guinea pigs are filled with the microbubble ultrasound contrast agent mixed the dye indicator and applied with the ultrasound; (2) the tympanic bullae of 9 guinea pigs are filled with the dye indicator and applied with the ultrasound; (3) the microbubble ultrasound contrast agent mixed the dye indicator is applied to the round windows of the remaining 27 guinea pigs, without applying the ultrasound, where the microbubble ultrasound contrast agent mixed the dye indicator is diffused into the round window membrane of the guinea pigs.

In the experiments of the present invention employs Sonoporation Gene Transfection System (ST2000V, NepaGene, Japan), with a probe size of 6 mm and the waveform of square waves. In the experiments, the ultrasound is operated at a frequency of 1 MHz, a duty cycle of 50%, energy of 3 W/cm², is applied for 1 minute. In the experiments, the probe is placed on the body surface facing the round window membrane with a distance of 5 mm.

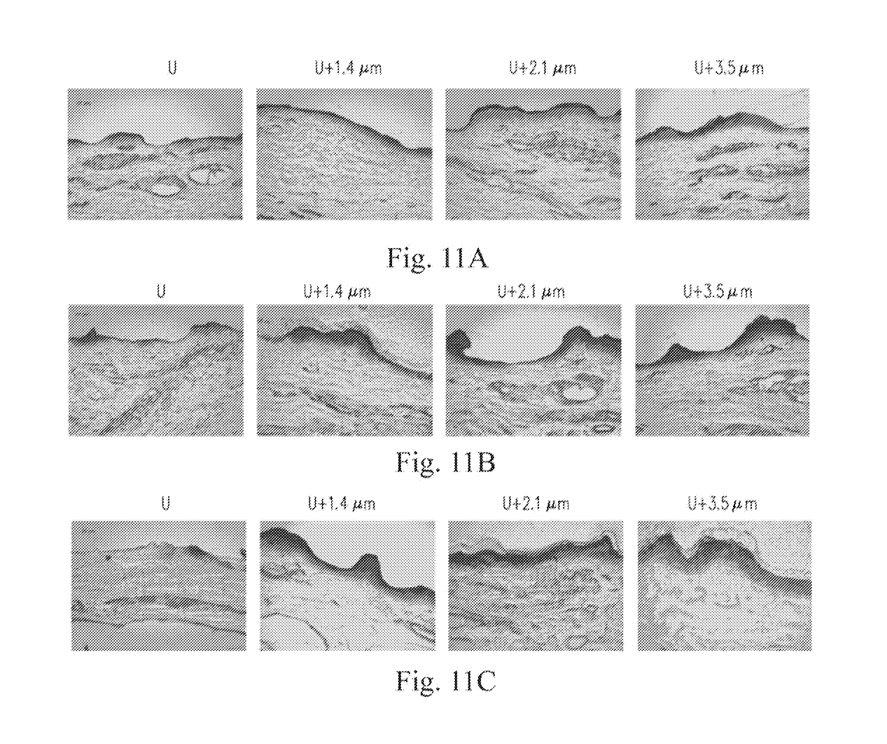

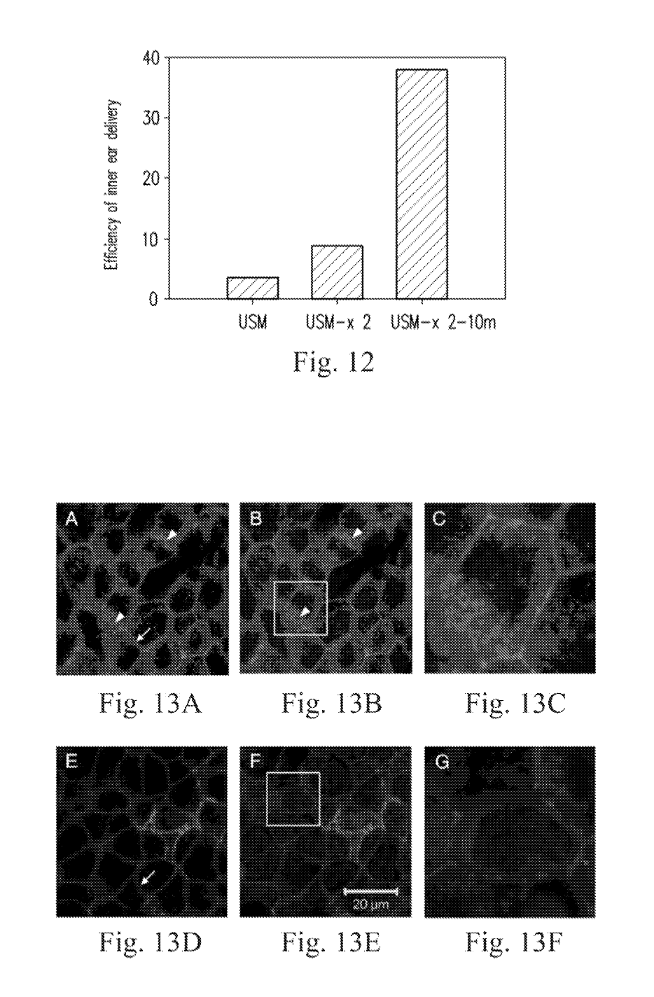

FIG. 12 shows the results of the delivery efficiency using different administration approaches of the microbubble contrast agent in the inner ear treatment experiments. USM refers to give the microbubble ultrasound contrast agent once and apply the ultrasound once, USM×2 refers to give the microbubble ultrasound contrast agent twice and apply the ultrasound twice, USM×2-10 m refers to give the microbubble ultrasound contrast agent twice and apply the ultrasound twice and stranded for 10 minutes. Compared to the control group of delivering the dye or drug into the inner ear through the diffusion effect, the experimental results indicate that the ultrasound used together with the microbubble ultrasound contrast agent can enhance the drug delivery efficiency. That is, the delivery efficiency of the administration approaches USM, USM×2, USM×2-10 m is respectively 3.5 times, 8.8 times, 37.9 times of that of the control group. In addition, in order to deliver gentamycin into the inner ear, the microbubbles ultrasound contrast agent of this invention is used along with the application of the ultrasound. By using such approach, the concentration of gentamycin delivered into the cochlear tissues is significantly higher than that of the control group without applying the ultrasound. Hence it is confirmed that the microbubble contrast agent can enhance the delivery of the chemical and promote the absorption of the drug or small molecules.

FIGS. 13A-13F show the delivery results of the green dye indicator entering into the round window membrane cells of the inner ear under different administration approaches. FIGS. 13A-136C show the delivery results of the experimental groups using the ultrasound microbubble contrast agent mixed with the green dye indicator and operated with the ultrasound. FIGS. 13D-13F show the delivery results of the control groups using the ultrasound microbubble contrast agent mixed with the green dye indicator but without applying the ultrasound (through the diffusion effect). Compared the results of little or no green dye entering into the round window membrane cells in FIGS. 13D-136F, the results of FIGS. 13A-13C show much more green dyes entering into the round window membrane cells.

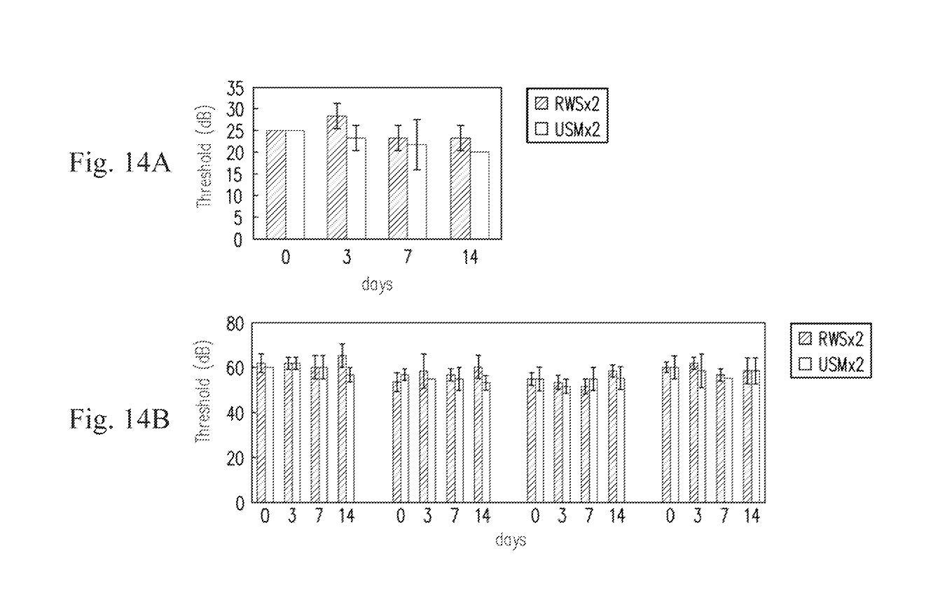

In addition, in order to verify whether the microbubble ultrasound contrast agent(s) of the present invention will do harm to the cells in the inner ear cochlea, the present invention also perform hearing threshold functional evaluation experiments on the guinea pigs experiencing the aforementioned animal tests. FIGS. 14A-14B show the results of the auditory brainstem response tests of the animals following the animal tests. The animals in the experimental group administrated with the drug and ultrasound (denoted as USM) or in the control group administrated with the drugs without ultrasound (denoted as RWS) further went through the auditory brainstem response tests on ticking sounds (FIG. 14A) and plosive sounds (FIG. 14B). The results show no difference between two groups in the hearing thresholds, indicating that the microbubble ultrasound contrast agents acting on the inner ear cochlea causes no harm to the cells in the auditory system.

The ultrasound applicable in the present invention is preferably a non-focusing type low-energy ultrasound, and its energy range is of the MI=0.2-0.4, compared to the FDA provisions for the medical ultrasound being below the MI of 1.9 or the ultrasound for ophthalmic uses being below the MI of 0.2, the energy range of the ultrasound applicable in the present invention is far below these ranges. Furthermore, the energy range of the ultrasound used in the present invention does not cause local temperature variations. In the experiments of the present invention, it is found that the temperature difference is only plus or minus 0.1 degree during the operation. Therefore, the energy range of the ultrasound used in the present invention will not have thermal effects.

It will be apparent to those skilled in the art that various modifications and variations can be made to the structure of the disclosure without departing from the scope or spirit of the disclosure. In view of the foregoing, it is intended that the disclosure cover modifications and variations of this disclosure provided they fall within the scope of the following claims and their equivalents.

What is claimed is:

1. An external use microbubble ultrasound contrast agent, comprising:
a medium, wherein the medium is in a form of an aqueous solution or a gel form; and
a plurality of microbubbles dispersed in the medium, wherein a concentration of the microbubbles ranges from 4×10⁸ to 2×10¹⁰ particles/ml.

2. The microbubble ultrasound contrast agent as claimed in claim 1, wherein a material of the microbubbles is selected from albumin, polymers, liposomes, copolymers or mixtures thereof or a combination of thereof, and the medium is selected from an isotonic saline solution, an agar gel, an aloe gel, a topical gel or a combination of thereof.

3. The microbubble ultrasound contrast agent as claimed in claim 2, wherein the material of the microbubbles further comprises hexose and/or pentose.

4. The microbubble ultrasound contrast agent as claimed in claim 3, wherein the hexose is dextrose.

5. The microbubble ultrasound contrast agent as claimed in claim 1, wherein the medium is a gel form medium and a content of the gel form medium is less than or equivalent to 0.2 percentages by weight of a total weight of the microbubble ultrasound contrast agent.

6. The microbubble ultrasound contrast agent as claimed in claim 1, wherein a particle size of the microbubbles ranges from 0.5 micrometers to 3.7 micrometers.

7. The microbubble ultrasound contrast agent as claimed in claim 1, further comprising a chemical or small molecules, wherein the chemical or the small molecules are percutaneously absorbed by a biological body.

8. A method of enhancing percutaneous absorption of a chemical or small molecules through a topical region of a biological body surface, comprising:
applying a microbubble ultrasound contrast agent to the topical region of the biological body surface, wherein the microbubble ultrasound contrast agent comprises a medium and a plurality of microbubbles dispersed in the medium, the medium is in a form of an aqueous solution or a gel form, and a material of the microbubbles is selected from albumin, polymers, liposomes, copolymers or mixtures thereof or a combination of thereof;
applying the chemical or the small molecules to the topical region; and
applying a mechanical oscillation wave source to be in direct contact with the topical region applied with the microbubble ultrasound contrast agent and the chemical or the small molecules, through mechanical waves generated by the mechanical oscillating energy source acting on the microbubbles, so as to increase the percutaneous absorption of the chemical or the small molecules.

9. The microbubble ultrasound contrast agent as claimed in claim 2, wherein the material of the microbubbles further comprises hexose and/or pentose.

10. The microbubble ultrasound contrast agent as claimed in claim 9, wherein the hexose is dextrose and the medium is an isotonic saline solution.

11. The method of claim 8, wherein a concentration of the microbubbles ranges from about 4×10⁸ to about 2×10¹⁰ particles/ml, relative to the total volume of the microbubble ultrasound contrast agent and the chemical or the small molecules.

12. The method of claim 11, further comprising using the chemical or the small molecules as a diluent to dilute the microbubble ultrasound contrast agent 2-1000 times.

13. The method of claim 8, wherein the steps of applying the microbubble ultrasound contrast agent and applying the chemical or the small molecules are performed separately.

14. The method of claim 8, wherein a particle size of the microbubbles ranges from 0.5 micrometers to 3.7 micrometers.

15. The method of claim 8, wherein the mechanical oscillation wave source includes an ultrasound source and/or a laser source.

16. A method of preparing a microbubble ultrasound contrast agent, comprising:
mixing a microbubble material with a medium to form a mixed solution;
applying an ultrasonic oscillating source oscillating the mixed solution for about 100 to about 140 seconds to form the microbubble ultrasound contrast agent comprising a plurality of microbubbles, wherein a particle size of the microbubbles ranges from 0.5 micrometers to 3.7 micrometers.

17. The method of claim 16, wherein a concentration of the microbubbles ranges from 4×10⁸ to 2×10¹⁰ particles/ml.

18. The method of claim 16, wherein the microbubble material is selected from albumin, polymers, liposomes, copolymers or mixtures thereof or a combination of thereof.

19. The method of claim 18, wherein the microbubble material further comprises pentose and/or hexose.

20. The method of claim 19, wherein the hexose is dextrose.

21. The method of claim 16, wherein the medium is isotonic saline solution.

22. The method of claim 16, wherein the microbubble material is albumin.

23. The method of claim 16, wherein the microbubbles comprise octafluoropropane (C₃F₈) inside the microbubbles.

24. The method of claim 16, wherein the microbubble material comprises albumin and the medium is isotonic saline solution, the mixed solution comprises albumin for from about 0.5 to about 1 wt %, and the formed microbubbles having average particle size for from about 0.5 to about 1 μm.

25. The method of claim 16, wherein the microbubble material comprises albumin or albumin and dextrose, the medium comprises isotonic saline solution, the mixed solution comprises albumin for from about 1 to about 1.5 wt %, or albumin for 1.32 wt % and the dextrose for from about 3 to about 7 wt %, or albumin for from about 0.5 to about 1 wt % and the dextrose for from about 8 to about 12 wt %, the formed microbubbles having average particle size for from about 1 to about 1.5 μm.

26. The method of claim 16, wherein the microbubble material comprises albumin and dextrose, the medium comprises isotonic saline solution, the mixed solution comprises albumin for 1.32 wt % and the dextrose for from about 8 to about 17 wt %, or albumin for from about 4.8 to about 5.2 wt % and the dextrose for from about 43 to about 47 wt %, the formed microbubbles having average particle size for from about 1.5 to about 2 μm.

27. The method of claim 16, wherein the microbubble material comprises albumin and dextrose, the medium comprises isotonic saline solution, the mixed solution comprises albumin for 1.32 wt % and the dextrose for from about 18 to about 32 wt %, or albumin for from about 1.8 to about 2.2 wt % and the dextrose for from about 8 to about 12 wt %, or albumin for from about 3.3 to about 3.7 wt % and the dextrose for from about 43 to about 47 wt %, the formed microbubbles having average particle size for from about 2 to about 2.5 μm.

28. The method of claim 16, wherein the microbubble material comprises albumin or albumin and dextrose, the medium comprises isotonic saline solution, the mixed solution comprises albumin for from about 1.8 to about 5.2 wt %, albumin for 1.32 wt % and the dextrose for from about 38 to about 42 wt %, albumin for from about 3.3 to about 3.7 wt % and the dextrose for from about 8 to about 12 wt %, or albumin for from about 1.8 to about 2.2 wt % and the dextrose for from about 43 to about 47 wt %, the formed microbubbles having average particle size for from about 2.5 to about 3 μm.

29. The method of claim 16, wherein the microbubble material comprises albumin and dextrose, the medium comprises isotonic saline solution, the mixed solution comprises albumin for 1.32 wt % and the dextrose for from about 43 to about 47 wt %, or albumin for from about 4.8 to about 5.2 wt % and the dextrose for from about 8 to about 12 wt %, the formed microbubbles having average particle size for from about 3 to about 3.5 μm.
